(19) United States
(12) Patent Application Publication
(10) Pub. No.: US 2025/0302648 A1
(43) Pub. Date: Oct. 2, 2025

(54) Stent Delivery Apparatus and Methods of Manufacture

(71) Applicant: ACCUMEDICAL BEIJING LTD., Beijing (CN)

(72) Inventors: Tai Dac Tieu, Fountain Valley, CA (US); Edver Bahena, Irvine, CA (US)

(73) Assignee: ACCUMEDICAL BEIJING LTD., Beijing (CN)

(21) Appl. No.: 18/623,361

(22) Filed: Apr. 1, 2024

(51) Int. Cl. A61F 2/95

(52) U.S. Cl. CPC A61F 2/95; A61F 2250/0098

(57) ABSTRACT

An improved stent delivery apparatus is disclosed and configured for having radiopaque properties without sacrificing the structural integrity or functionality of the apparatus itself. In at least one embodiment, the apparatus provides a plurality of individual strands braided together to form a self-expanding stent delivery structure having a proximal end and an opposing distal end, the delivery structure forming an elongated primary shape capable of being inserted into a catheter. An at least one radiopaque marker is coaxially positioned within and secured to a circumferential inner surface of the delivery structure. Accordingly, the delivery structure has a primary outer diameter that is substantially uniform along an entire length of the delivery structure.

### RELATED APPLICATIONS

Not applicable.

### BACKGROUND

The subject of this patent application relates generally to stents, and more particularly to an improved stent delivery apparatus and associated methods of manufacture.

Applicant hereby incorporates herein by reference any and all patents and published patent applications cited or referred to in this application.

By way of background, stents are tubular devices that are delivered into a patient's vascular system via a catheter or similar delivery device, and are used to treat a variety of different conditions, including stenosis and aneurysms. When a stent is deployed in a generally linear portion of a vessel, stent delivery is typically straightforward. For example, a distal end of a delivery catheter is advanced near a desired delivery location and the outer sheath is withdrawn, allowing the stent to expand within the vessel. Even in these linear vessel situations, however, stent delivery can still be challenging due to variables such as the size of the stent, size of the delivery catheter, and size of the vessel. Often, when the stent must be deployed at or near a tortuous or heavily curved portion of a vessel, a self-expanding stent may have difficulty properly deploying.

To assist in accurate placement of the stent, it is useful to visually monitor the stent as it advances through a vessel. Accordingly, as illustrated in FIG. 1, some prior art stent delivery devices 20 incorporate one or more radiopaque marker bands 22 positioned circumferentially about an outer surface 24 of the stent delivery device 20. As used herein, the term “radiopaque” is utilized for its normal meaning of being radiodense, that is, formed of one or more materials (such as platinum, chromium, cobalt, tantalum, tungsten, nitinol, gold, silver, stainless steel or alloys thereof, for example) which inhibit the passage of electromagnetic radiation to increase visibility during imaging. However, because such prior art marker bands 22 are positioned circumferentially about the outer surface 24 of the stent delivery device 20, the stent delivery device 20 has a relatively larger outer diameter at each marker band 22 location. Additionally, each of the opposing terminal ends 26 of each marker band 22 (i.e., the locations where the diameter of the stent delivery device 20 changes by virtue of the marker bands 22) creates a rigid shoulder that can potentially catch on the deployed stent, catheter, the vessel, or other components as the stent delivery device 20 is being pushed or pulled. Another downside with such prior art marker bands 22 being positioned circumferentially about the outer surface 24 of the stent delivery device 20 is that such placement limits the potential shapes and configurations of the marker bands 22.

Accordingly, there remains a need for an improved stent delivery device having radiopaque properties without sacrificing the structural integrity or functionality of the stent delivery system and the stent itself. Aspects of the present invention fulfill these needs and provide further related advantages as described in the following summary.

It should be noted that the above background description includes information that may be useful in understanding aspects of the present invention. It is not an admission that any of the information provided herein is prior art or relevant to the presently claimed invention, or that any publication specifically or implicitly referenced is prior art.

### SUMMARY

Aspects of the present invention teach certain benefits in construction and use which give rise to the exemplary advantages described below.

The present invention solves the problems described above by providing an improved stent delivery apparatus configured for having radiopaque properties without sacrificing the structural integrity or functionality of the apparatus itself. In at least one embodiment, the apparatus provides a plurality of individual strands braided together to form a self-expanding stent delivery structure having a proximal end and an opposing distal end, the delivery structure forming an elongated primary shape capable of being inserted into a catheter. An at least one radiopaque marker is coaxially positioned within and secured to a circumferential inner surface of the delivery structure. Accordingly, the delivery structure has a primary outer diameter that is substantially uniform along an entire length of the delivery structure.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is a partial side elevational view of an exemplary prior art stent delivery device;

FIG. 2 is a partial side elevational view of an exemplary improved stent delivery apparatus, in accordance with at least one embodiment;

FIG. 3 is an end elevational view thereof, in accordance with at least one embodiment;

FIG. 4 is a partial side elevational view of a further exemplary improved stent delivery apparatus, in accordance with at least one embodiment;

FIG. 5 is a detailed view of the section defined by line 5 of FIG. 4;

FIG. 6 is a perspective view thereof, in accordance with at least one embodiment;

FIG. 7 is an end elevational view thereof, in accordance with at least one embodiment;

FIG. 8 is a further partial side elevational view of the stent delivery apparatus of FIG. 4, in accordance with at least one embodiment;

FIG. 9 is a partial side elevational view of a still further exemplary stent delivery apparatus, with a delivery structure of the apparatus omitted for illustrative purposes, in accordance with at least one embodiment;

FIG. 10 is an end elevational view thereof, in accordance with at least one embodiment; and

FIGS. 11 and 12 are perspective views of further exemplary markers, in accordance with at least one embodiment.

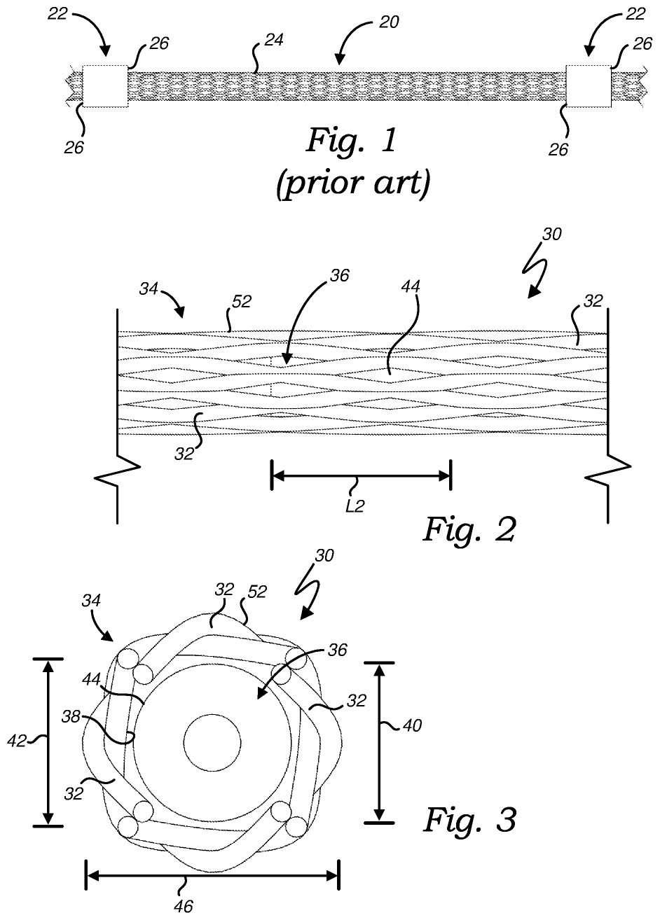

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

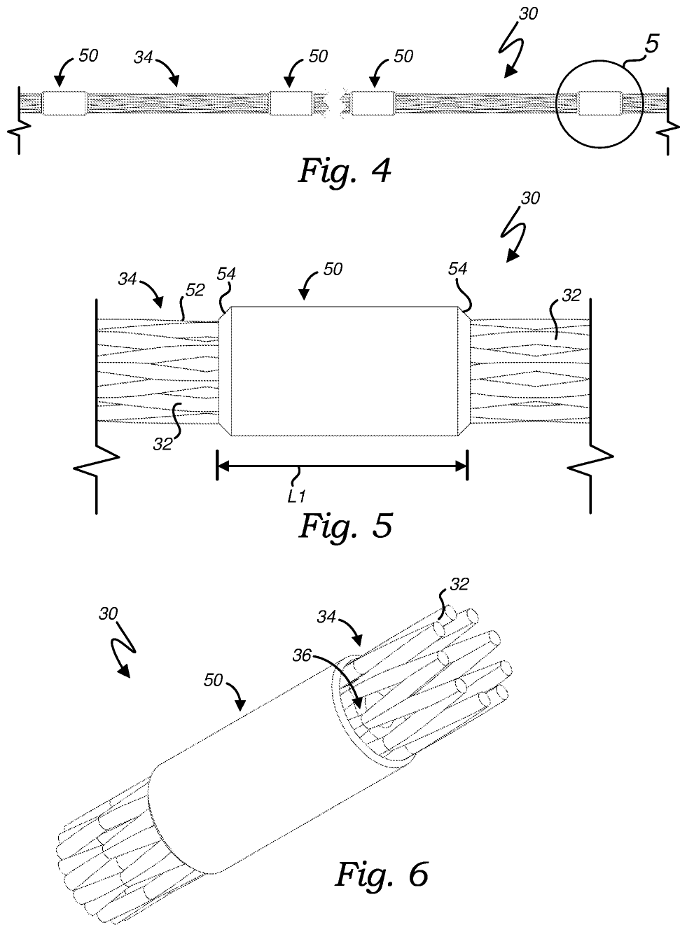

Turning now to FIG. 2, there is shown a partial side elevational view of an exemplary embodiment of an improved stent delivery apparatus 30. In at least one embodiment, the apparatus 30 provides a plurality of individual strands 32 braided together to form a self-expanding stent delivery structure 34 having an elongated primary shape (as best illustrated in FIG. 4). At the outset, it should be noted that the term “strand” is intended in its broadest meaning to include a wire, a fiber, a filament, or other single elongated member. In at least one embodiment, a proximal end of the delivery structure 34 is removably engageable with a delivery mechanism (not shown), such as a wire for example, for moving the delivery structure 34 through a catheter (not shown). In at least one embodiment, an opposing distal end of the delivery structure 34 provides an end cap (not shown) for maintaining the braided arrangement of the strands 32, similar to the end cap shown and described in U.S. Pat. No. 11,134,954, the contents of which are hereby incorporated herein by reference. In at least one such embodiment, the end cap is constructed out of one or more radiopaque materials to facilitate positioning of the delivery structure 34 at a desired location within a blood vessel or another target area within a vascular system of a patient (hereinafter generally referred to as a “target site” for simplicity purposes). In at least one alternate embodiment, one or both of the proximal and distal ends of the delivery structure 34 are simply closed, sealed or otherwise finished so as to maintain the braided arrangement of the strands 32.

In at least one embodiment, given that the delivery structure 34 is comprised of a plurality of strands 32 braided together, the properties of the delivery structure 34 may be selectively tailored to fit the requirements of the medical procedure for which the apparatus 30 is to be used. For example, in at least one embodiment, one or more individual strands 32 may be comprised of materials that are different from the other strands 32 of the delivery structure 34. In at least one such embodiment, at least one of the strands 32 is comprised of one or more radiopaque materials, while the remaining strands 32 are comprised of one or more non-radiopaque materials (such as fiber, plastic, polymers, multi-layer composites or other biocompatible materials, for example). In this way, the radiopacity of the delivery structure 34 may be selectively tailored (by including a lower or higher quantity of radiopaque strands 32) while also maintaining the requisite structural integrity for necessary shape retention, as discussed further below. In at least one further embodiment, one or more individual strands 32 may be comprised of both radiopaque and non-radiopaque materials. In at least one further embodiment, one or more individual strands 32 may be comprised of drawn filled tube bi-materials or compounded bimetals. Those skilled in the art will understand that other suitably bio-compatible materials may be used so long as they possess appropriate mechanical properties. It should also be noted that the specific braiding patterns illustrated in the accompanying figures are merely exemplary and are being shown and described for illustrative purposes only. In further embodiments, the strands 32 of the delivery structure 34 may be braided using any braid, knit, or weave patterns now known or later developed (the terms “braid” and “braided” as used herein intended to be all-encompassing for simplicity purposes)—including but in no way limited to 1-over-1-under-1, 1-over-2-under-2, 2-over-2-under-2, etc.—so long as the apparatus 30 is able to substantially carry out the functionality described herein. Additionally, braid densities and braid gaps depicted in the drawings are merely exemplary as well. In still further embodiments, the delivery structure 34 may utilize any other braid densities, and any braid gaps may take on any other sizes, shapes, quantities, dimensions or patterns, now known or later developed, so long as the apparatus 30 is able to substantially carry out the functionality described herein. Similarly, the strands 32 themselves may each take on any other sizes, quantities or dimensions, now known or later developed, so long as the apparatus 30 is able to substantially carry out the functionality described herein. For example, in at least one embodiment, one or more of the strands 32 may have a round shape, a flat shape, a square shape, a hexagonal shape, an oval shape, a ribbon shape, a hollow/tubular shape, etc. in cross-section.

In at least one embodiment, the elongated primary shape of the delivery structure 34 is suitable for insertion through the catheter into the vascular system with minimal discomfort to the patient. In at least one such embodiment, while in the primary shape, the delivery structure 34 has the shape of an elongated bundle of braided strands 32, as discussed above. Once the delivery structure 34 has reached the target site, with the stent (not shown) still coaxially positioned on an outer surface 52 of the delivery structure 34, the delivery structure 34 will expand to a pre-determined secondary shape (an example of which is illustrated in FIG. 8) to enhance stent opening and wall apposition. Accordingly, the sizes, shapes and dimensions of each of the primary shapes and secondary shapes depicted in the accompanying figures are merely exemplary and shown for illustrative purposes.

In at least one embodiment, as best illustrated in FIGS. 2 and 3, the apparatus 30 provides an at least one radiopaque marker 36 coaxially positioned within the delivery structure 34. In at least one such embodiment, the apparatus 30 provides a plurality of markers 36 coaxially positioned within the delivery structure 34 and longitudinally spaced apart from one another along a length of the delivery structure 34. In at least one embodiment, each of the at least one marker 36 is secured to a circumferential inner surface 38 of the delivery structure 34. Accordingly, in at least one such embodiment, the at least one marker 36 has an outer diameter 40 that is relatively smaller than a primary inner diameter 42 of the delivery structure 34 (i.e., the inner diameter of the delivery structure 34 when the delivery structure 34 is in its elongated primary shape, as depicted in FIG. 4 for example). In at least one embodiment, the outer diameter 40 of the at least one marker 36 approximates the primary inner diameter 42 of the delivery structure 34. In at least one embodiment, a circumferential outer surface 44 of the at least one marker 36 is permanently secured to the inner surface 38 of the delivery structure 34 using welding, adhesive, or any other material or technique (now known or later developed) capable of creating a permanent engagement between the outer surface 44 of the at least one marker 36 and the inner surface 38 of the delivery structure 34. Thus, with the at least one marker 36 positioned within the delivery structure 34, a primary outer diameter 46 (i.e., the outer diameter of the delivery structure 34 when the delivery structure 34 is in its elongated primary shape, as depicted in FIG. 4 for example) of the delivery structure 34 remains unchanged, such that the primary outer diameter 46 is substantially uniform along an entire length of the delivery structure 34. In other words, unlike the known prior art stents 20 discussed above, the at least one internally positioned marker 36 does not create any rigid external edges or shoulder-like protrusions that could potentially catch on the deployed stent, catheter, the vessel, or other components as the delivery structure 34 is being pushed or pulled.

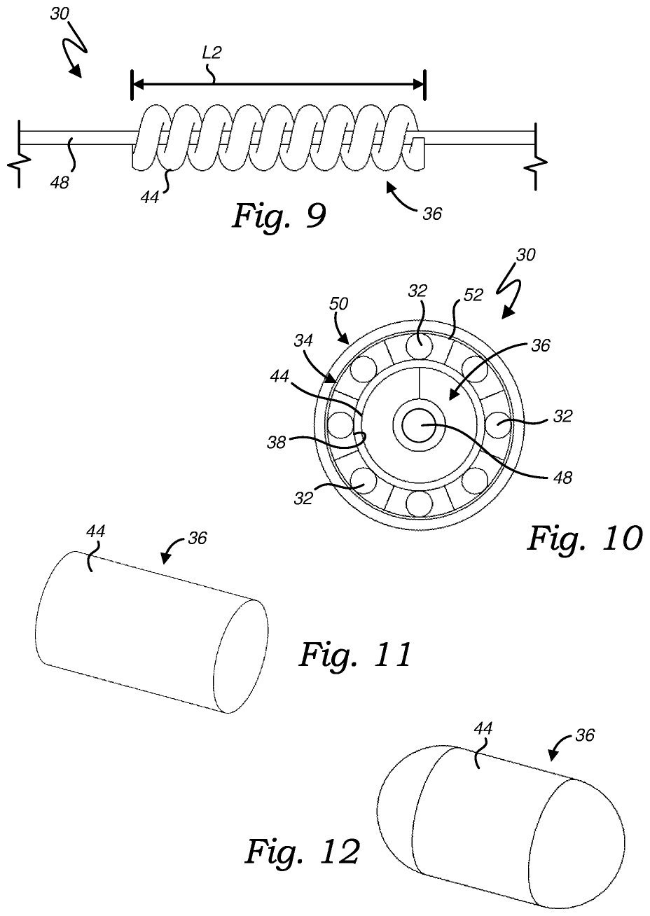

In at least one embodiment, the at least one marker 36 is constructed out of one or more radiopaque materials to facilitate positioning of the delivery structure 34 at a desired location relative to the target site within a vessel. In at least one such embodiment, the at least one marker 36 is constructed out of a tungsten-loaded polymer. In further embodiments, the at least one marker 36 may be constructed out of platinum, chromium, cobalt, tantalum, nitinol, gold, silver, bismuth subcarbonate, barium sulfate, bismuth oxychloride, bismuth trioxide, stainless steel or alloys thereof, or any other radiopaque material (or combination of materials) now known or later developed. In at least one alternate embodiment, the at least one marker 36 is constructed out of one or more non-radiopaque materials, with said at least one marker 36 instead being permanently secured to the inner surface 38 of the delivery structure 34 using an adhesive material (such as glue or epoxy, for example) that contains radiopaque materials. Accordingly, the term “radiopaque marker” as used herein is intended to include embodiments where the at least one marker 36 itself is constructed out of one or more radiopaque materials, as well as embodiments where the at least one marker 36 itself is constructed out of non-radiopaque materials but the adhesive used to secure said at least one marker 36 to the inner surface of the delivery structure 34 contains radiopaque materials. In at least one embodiment, the at least one marker 36 is substantially tubular (i.e., hollow) in structure. In at least one such embodiment, as best illustrated in FIG. 3, the at least one marker 36 has a substantially cylindrical shape. In at least one alternate such embodiment, as best illustrated in FIGS. 9 and 10, the at least one marker 36 has a substantially coil/spiral shape, thereby increasing the flexibility of the at least one marker 36. In at least one such embodiment, as best illustrated in FIG. 9, where the at least one marker 36 is substantially tubular in structure, the apparatus 30 further provides an at least one elongate guide wire 48 coaxially positioned within and extending longitudinally through the at least one marker 36 along with the delivery structure 34. In at least one alternate embodiment, as best illustrated in FIGS. 11 and 12, the at least one marker 36 is solid (i.e., not substantially tubular in structure). In still further embodiments, the at least one marker 36 may take on any other sizes, shapes, dimensions, configurations, quantities and/or relative positions now known or later developed-dependent at least in part on the specific context in which the apparatus 30 is to be utilized-so long as the apparatus 30 is able to substantially carry out the functionality described herein. In at least one embodiment, where the apparatus 30 provides a plurality of markers 36, each of the markers 36 has the same size, shape and dimensions. In at least one alternate embodiment, where the apparatus 30 provides a plurality of markers 36, one or more of the markers 36 may have a different, size, shape and/or dimensions.

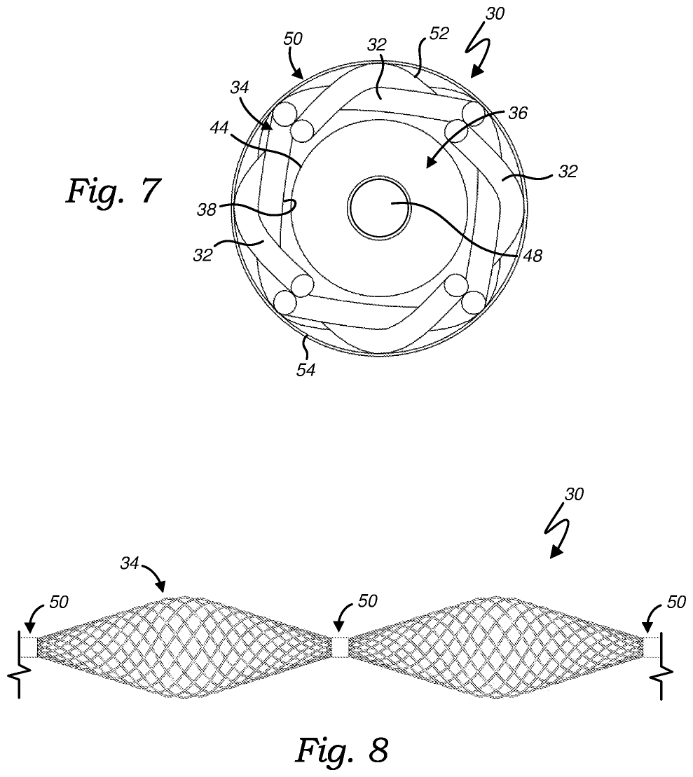

In at least one embodiment, as best illustrated in FIGS. 4-8, the apparatus 30 further provides an at least one substantially tubular protective outer barrier 50 circumferentially engaged about the outer surface 52 of the delivery structure 34, in substantially the same position as the at least one marker 36, such that the at least one outer barrier 50, delivery structure 34, and at least one marker 36 are concentrically positioned. In at least one such embodiment, the at least one outer barrier 50 has a barrier length L1 (FIG. 5) that is greater than or equal to a marker length L2 of the corresponding at least one marker 36 (FIG. 2), such that the at least one outer barrier 50 substantially covers the marker 36 (along with the corresponding length of the delivery structure 34 that is sandwiched therebetween). In at least one alternate embodiment, the at least one outer barrier 50 has a barrier length L1 that is less than the marker length L2 of the corresponding at least one marker 36. In at least one embodiment, the at least one outer barrier 50 is permanently secured to the outer surface 52 of the delivery structure 34 using welding, adhesive, or any other material or technique (now known or later developed) capable of creating a permanent engagement between the at least one outer barrier 50 and the outer surface 52 of the delivery structure 34. In at least one embodiment, the at least one outer barrier 50 is constructed out of a heat-shrink material, such as PET or PEBAX, for example. However, in further embodiments, the at least one outer barrier 50 may be constructed out of any other material (or combination of materials) now known or later developed-dependent at least in part on the specific context in which the apparatus 30 is to be utilized-so long as the apparatus 30 is able to substantially carry out the functionality described herein. In at least one embodiment, opposing barrier ends 54 of the at least one outer barrier 50 are tapered so as to assist in preventing the apparatus 30 from catching on the catheter, the vessel, or other components as the delivery structure 34 is being pushed or pulled, which could damage said components or even the delivery structure 34 itself. In further embodiments, the at least one outer barrier 50 may take on any other sizes, shapes, dimensions, configurations, quantities and/or relative positions now known or later developed-dependent at least in part on the specific context in which the apparatus 30 is to be utilized-so long as the apparatus 30 is able to substantially carry out the functionality described herein.

In at least one embodiment, the apparatus 30 is manufactured by temporarily positioning the at least one marker 36 on an elongate support wire, then moving the at least one marker 36 (via the support wire) coaxially through the delivery structure 34 until the at least one marker 36 is in the desired position within the delivery structure 34. The outer surface 44 of the at least one marker 36 is then secured to the inner surface 38 of the stent delivery structure 34 as described above, and the support wire is disengaged from said marker 36. In at least one alternate embodiment, the support wire is left is place and secured to said marker 36 using welding, adhesive, or any other material or technique (now known or later developed) capable of creating an engagement between the support wire and said marker 36. In at least one embodiment, an outer barrier 50 is then positioned overtop of the at least one marker 36 and the corresponding length of the delivery structure 34, and the outer barrier 50 is then secured to the outer surface 52 of the delivery structure 34 as described above. In at least one embodiment, these steps are repeated for each of the at least one marker 36. In at least one alternate embodiment, where the apparatus 30 provides a plurality of markers 36, two or more of the markers 36 are positioned simultaneously on the support wire a desired distance apart from one another (based on the distance that said markers 36 are to be spaced apart when engaged with the delivery structure 34), thereby allowing the above steps to be carried out for a plurality of markers 36 simultaneously. In still further alternate embodiments, any other methods for manufacturing the apparatus 30 (now known or later developed) may be utilized, so long as the apparatus 30 is able to substantially carry out the functionality described herein.

Aspects of the present specification may also be described as the following embodiments:

1. A stent delivery apparatus comprising: a plurality of individual strands braided together to form a self-expanding stent delivery structure having a proximal end and an opposing distal end, the delivery structure forming an elongated primary shape capable of being inserted into a catheter; and an at least one radiopaque marker coaxially positioned within and secured to a circumferential inner surface of the delivery structure; whereby, the delivery structure has a primary outer diameter that is substantially uniform along an entire length of the delivery structure.

2. The stent delivery apparatus according to embodiment 1, wherein the proximal end of the delivery structure is removably engageable with a delivery mechanism for selectively moving the delivery structure through a catheter.

3. The stent delivery apparatus according to embodiments 1-2, wherein the distal end of the delivery structure provides an end cap for maintaining the braided arrangement of the strands of the delivery structure.

4. The stent delivery apparatus according to embodiments 1-3, wherein the end cap is constructed out of an at least one radiopaque material.

5. The stent delivery apparatus according to embodiments 1-4, wherein at least one of the strands of the delivery structure is comprised of an at least one material that is different from the other strands of the delivery structure.

6. The stent delivery apparatus according to embodiments 1-5, wherein at least one of the strands of the delivery structure is comprised of an at least one radiopaque material.

7. The stent delivery apparatus according to embodiments 1-6, wherein at least one of the strands of the delivery structure is comprised of an at least one non-radiopaque material.

8. The stent delivery apparatus according to embodiments 1-7, wherein at least one of the strands of the delivery structure is comprised of both radiopaque and non-radiopaque materials.

9. The stent delivery apparatus according to embodiments 1-8, further comprising a plurality of markers coaxially positioned within the delivery structure and longitudinally spaced apart from one another along a length of the delivery structure.

10. The stent delivery apparatus according to embodiments 1-9, wherein the at least one marker has an outer diameter that is relatively smaller than a primary inner diameter of the delivery structure.

11. The stent delivery apparatus according to embodiments 1-10, wherein the at least one marker has an outer diameter that approximates a primary inner diameter of the delivery structure.

12. The stent delivery apparatus according to embodiments 1-11, wherein a circumferential outer surface of the at least one marker is permanently secured to the inner surface of the delivery structure.

13. The stent delivery apparatus according to embodiments 1-12, wherein the at least one marker is constructed out of an at least one radiopaque material.

14. The stent delivery apparatus according to embodiments 1-13, wherein the at least one marker is substantially tubular in structure.

15. The stent delivery apparatus according to embodiments 1-14, wherein the at least one marker has a substantially cylindrical shape.

16. The stent delivery apparatus according to embodiments 1-15, wherein the at least one marker has a substantially coil shape.

17. The stent delivery apparatus according to embodiments 1-16, further comprising an at least one elongate guide wire coaxially positioned within and extending longitudinally through the at least one marker along with the delivery structure.

18. The stent delivery apparatus according to embodiments 1-17, wherein the at least one marker is not tubular in structure.

19. The stent delivery apparatus according to embodiments 1-18, further comprising an at least one substantially tubular protective outer barrier circumferentially engaged about an outer surface of the delivery structure, in substantially the same position as the at least one marker, such that the at least one outer barrier, delivery structure, and at least one marker are concentrically positioned.

20. The stent delivery apparatus according to embodiments 1-19, wherein each of the at least one outer barrier has a barrier length that is greater than or equal to a marker length of the corresponding at least one marker over which said outer barrier is positioned, such that said outer barrier substantially covers said marker.

21. The stent delivery apparatus according to embodiments 1-20, wherein the at least one outer barrier is permanently secured to the outer surface of the delivery structure.

22. The stent delivery apparatus according to embodiments 1-21, wherein the at least one outer barrier is constructed out of a heat-shrink material.

23. The stent delivery apparatus according to embodiments 1-22, wherein opposing ends of the at least one outer barrier are tapered.

24. A stent delivery apparatus comprising: a plurality of individual strands braided together to form a self-expanding stent delivery structure having a proximal end and an opposing distal end, the delivery structure forming an elongated primary shape capable of being inserted into a catheter; and an at least one substantially coil shaped radiopaque marker coaxially positioned within the delivery structure, the at least one marker having a circumferential outer surface that is permanently secured to a circumferential inner surface of the delivery structure; whereby, the delivery structure has a primary outer diameter that is substantially uniform along an entire length of the delivery structure.

25. A stent delivery apparatus comprising: a plurality of individual strands braided together to form a self-expanding delivery structure having a proximal end and an opposing distal end, the delivery structure forming an elongated primary shape capable of being inserted into a catheter; an at least one radiopaque marker coaxially positioned within and secured to a circumferential inner surface of the delivery structure; and an at least one substantially tubular protective outer barrier circumferentially engaged about an outer surface of the delivery structure, in substantially the same position as the at least one marker, such that the at least one outer barrier, delivery structure, and at least one marker are concentrically positioned; whereby, the delivery structure has a primary outer diameter that is substantially uniform along an entire length of the delivery structure.

In closing, regarding the exemplary embodiments of the present invention as shown and described herein, it will be appreciated that an improved stent delivery apparatus and associated methods of manufacture are disclosed and configured for having radiopaque properties without sacrificing the structural integrity or functionality of the apparatus itself. Because the principles of the invention may be practiced in a number of configurations beyond those shown and described, it is to be understood that the invention is not in any way limited by the exemplary embodiments, but is generally directed to a stent delivery apparatus and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the terms “about” and “approximately.” As used herein, the terms “about” and “approximately” mean that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art, or at least encompassing a range of plus or minus ten percent above and below the value of the stated characteristic, item, quantity, parameter, property, or term.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators-such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter.

Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

Any claims intended to be treated under 35 U.S.C. § 112 (f) will begin with the words “means for,” but use of the term “for” in any other context is not intended to invoke treatment under 35 U.S.C. § 112 (f). Accordingly, Applicant reserves the right to pursue additional claims after filing this application, in either this application or in a continuing application.

It should be understood that any methods disclosed herein, along with the order in which the respective elements of any such method are performed, are purely exemplary. Depending on the implementation, they may be performed in any order or in parallel, unless indicated otherwise in the present disclosure.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A stent delivery apparatus comprising:
a plurality of individual strands braided together to form a self-expanding stent delivery structure having a proximal end and an opposing distal end, the delivery structure forming an elongated primary shape capable of being inserted into a catheter; and
an at least one radiopaque marker coaxially positioned within and secured to a circumferential inner surface of the delivery structure;
whereby, the delivery structure has a primary outer diameter that is substantially uniform along an entire length of the delivery structure.

2. The stent delivery apparatus of claim 1, wherein the distal end of the delivery structure provides an end cap for maintaining the braided arrangement of the strands of the delivery structure.

3. The stent delivery apparatus of claim 2, wherein the end cap is constructed out of an at least one radiopaque material.

4. The stent delivery apparatus of claim 1, wherein at least one of the strands of the delivery structure is comprised of an at least one radiopaque material.

5. The stent delivery apparatus of claim 1, further comprising a plurality of markers coaxially positioned within the delivery structure and longitudinally spaced apart from one another along a length of the delivery structure.

6. The stent delivery apparatus of claim 1, wherein the at least one marker has an outer diameter that is relatively smaller than a primary inner diameter of the delivery structure.

7. The stent delivery apparatus of claim 1, wherein the at least one marker has an outer diameter that approximates a primary inner diameter of the delivery structure.

8. The stent delivery apparatus of claim 1, wherein a circumferential outer surface of the at least one marker is permanently secured to the inner surface of the delivery structure.

9. The stent delivery apparatus of claim 1, wherein the at least one marker is substantially tubular in structure.

10. The stent delivery apparatus of claim 9, wherein the at least one marker has a substantially cylindrical shape.

11. The stent delivery apparatus of claim 9, wherein the at least one marker has a substantially coil shape.

12. The stent delivery apparatus of claim 9, further comprising an at least one elongate guide wire coaxially positioned within and extending longitudinally through the at least one marker along with the delivery structure.

13. The stent delivery apparatus of claim 1, wherein the at least one marker is not tubular in structure.

14. The stent delivery apparatus of claim 1, further comprising an at least one substantially tubular protective outer barrier circumferentially engaged about an outer surface of the delivery structure, in substantially the same position as the at least one marker, such that the at least one outer barrier, delivery structure, and at least one marker are concentrically positioned.

15. The stent delivery apparatus of claim 14, wherein each of the at least one outer barrier has a barrier length that is greater than or equal to a marker length of the corresponding at least one marker over which said outer barrier is positioned, such that said outer barrier substantially covers said marker.

16. The stent delivery apparatus of claim 14, wherein the at least one outer barrier is permanently secured to the outer surface of the delivery structure.

17. The stent delivery apparatus of claim 14, wherein the at least one outer barrier is constructed out of a heat-shrink material.

18. The stent delivery apparatus of claim 14, wherein opposing ends of the at least one outer barrier are tapered.

19. A stent delivery apparatus comprising:
a plurality of individual strands braided together to form a self-expanding stent delivery structure having a proximal end and an opposing distal end, the delivery structure forming an elongated primary shape capable of being inserted into a catheter; and
an at least one substantially coil shaped radiopaque marker coaxially positioned within the delivery structure, the at least one marker having a circumferential outer surface that is permanently secured to a circumferential inner surface of the delivery structure;
whereby, the delivery structure has a primary outer diameter that is substantially uniform along an entire length of the delivery structure.

20. A stent delivery apparatus comprising:
a plurality of individual strands braided together to form a self-expanding stent delivery structure having a proximal end and an opposing distal end, the delivery structure forming an elongated primary shape capable of being inserted into a catheter;
an at least one radiopaque marker coaxially positioned within and secured to a circumferential inner surface of the delivery structure; and
an at least one substantially tubular protective outer barrier circumferentially engaged about an outer surface of the delivery structure, in substantially the same position as the at least one marker, such that the at least one outer barrier, delivery structure, and at least one marker are concentrically positioned;
whereby, the delivery structure has a primary outer diameter that is substantially uniform along an entire length of the delivery structure.
